(19) United States
(12) Patent Application Publication
(10) Pub. No.: US 2009/0074815 A1
(43) Pub. Date: Mar. 19, 2009

(54) Immunomodulator Compounds as Vaccine Enhancers

(75) Inventors: Cynthia W. Tuthill, Menlo Park, CA (US); Alfred R. Rudolph, Los Altos Hills, CA (US)

Correspondence Address: ROTHWELL, FIGG, ERNST & MANBECK, P.C., 1425 K STREET, N.W., SUITE 800, WASHINGTON, DC, 20005, US

(73) Assignee: SCICLONE PHARMACEUTICALS, INC., Foster City, CA (US)

(21) Appl. No.: 11/912,266

(22) PCT Filed: Apr. 21, 2006
(86) PCT No.: PCT/US06/15047

§ 371 Date: Sep. 29, 2008

Related U.S. Application Data
(60) Provisional application No. 60/673,790, filed on Apr. 22, 2005.
(60) Provisional application No. 60/678,187, filed on May 6, 2005.
(60) Provisional application No. 60/765,761, filed on Feb. 7, 2006.

(51) Int. Cl. A61K 39/145; A61K 39/00; A61K 39/29; A61K 39/12; A61K 39/275

(52) U.S. Cl. 424/209.1; 424/184.1; 424/226.1; 424/227.1; 424/228.1; 424/204.1; 424/232.1

(57) ABSTRACT

A vaccination method utilizes a pharmaceutical combination for enhancing vaccine effectiveness. The method utilizes an immune response-triggering vaccine capable of stimulating production in an immunodefficicent animal of antibodies to a disease-causing agent foreign to the animal. As an adjuvant, a vaccine effectiveness-enhancing amount of an immunomodulator compound is administered, which enhances production and affinity of the antibodies in the animal, in response to the vaccine.

### CROSS-REFERENCE TO RELATED APPLICATIONS

This application claims the benefit of U.S. Provisional Application Ser. Nos. 60/673,790, filed Apr. 22, 2005; 60/678,187, filed May 6, 2005; and 60/765,761, filed Feb. 7, 2006, which are incorporated herein.

### FIELD OF THE INVENTION

The present invention relates to the field of vaccines.

### DESCRIPTION OF THE BACKGROUND ART

Humans, livestock and pets often are vaccinated to prevent disease, or reduce the severity of disease. Vaccination results in the production of antibodies, which are serum proteins capable of binding specifically to antigen substances used in the vaccine. This humoral response involves the selection of specific lines of B lymphocytes, and the proliferation and differentiation of the selected cells to yield clones of antibody-producing plasma cells.

Antibody production reaches a peak within several weeks after immunization, and then gradually declines. Because of a constant turnover of serum proteins, the decline in antibody production is accompanied by a corresponding decline in the circulating level of antibodies. However, if the patient is challenged again with the same antigen, a new response curve is initiated more rapidly and more intensely than the first one. This is called a secondary, booster, or anamnestic response, and in healthy patients results in much higher antibody levels with higher affinity to the antigen than the first exposure, or primary immunization. The increased rate of antibody synthesis is the result of an increased number of antibody-producing plasma cells. These cells are scarce in the lymph nodes of the unimmunized patient, which contain mostly small lymphocytes. However, in healthy patients, plasma cells constitute up to 3% of the total lymph node cells after a primary immunization, and as much as 30% of the lymph node cells after a secondary immunization.

The secondary response is said to be due to immunological memory. That is, the healthy organism is able to “remember” its prior exposure to the antigen, and react more promptly and efficiently the second time it is exposed, even if the amount of specific antibodies in the serum has declined to a very low level in the meantime.

Certain conditions such as aging, malnutrition, drug addiction, alcoholism, and certain disease states such as diabetes and AIDS, lead to immunodeficiency, in which many immune responses are quenched and vaccination is of reduced effectiveness. Thus, there remains a need in the art for improved vaccination techniques, particularly among the elderly or otherwise immunodeficient.

### SUMMARY OF THE INVENTION

In one embodiment the present invention provides a pharmaceutical combination that may be utilized in a vaccination method to enhance vaccine effectiveness. The pharmaceutical combination comprises an immune response-triggering vaccine capable of stimulating production in an immunodeficient animal of antibodies to a disease-causing agent foreign to the animal and a vaccine effectiveness-enhancing amount of an immunomodulator compound, which enhances production and affinity of the antibodies in the animal in response to the vaccine. The vaccine and the immunomodulator may be administered separately or together.

In a preferred method, the vaccination method comprises administering to an immunodeficient animal a first dose of an immune response-triggering vaccine capable of stimulating production in an animal of antibodies to a disease-causing agent foreign to the animal. In a further preferred embodiment a vaccine effectiveness-enhancing amount of an immunomodulator compound may be administered to enhance production of the antibodies in the animal in response to the vaccine at the time of or in the time period immediately after and about 2 months after administration of said first dose. Alternatively, a booster dose of the vaccine, along with a vaccine effectiveness-enhancing amount of the immunomodulator compound may be administered to enhance effectiveness of the vaccine in said animal. In an alternative embodiment the pharmaceutical composition may be used to enhance the production of antibodies in an animal in response to administration of decreased quantities of vaccine.

Immunomodulator compounds in accordance with the present invention, comprise immunomodulator compounds of Formula A:

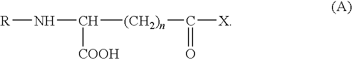

In Formula A, n is 1 or 2, R is hydrogen, acyl, alkyl or a peptide fragment, and X is an aromatic or heterocyclic amino acid or a derivative thereof. Preferably, X is L-tryptophan or D-tryptophan.

### BRIEF DESCRIPTION OF THE FIGURES

FIG. 1 provides the results of assays which validate the low-dose aerosol infection of mice with BCG.

FIG. 2 provides a graphical representation of lung bacterial burden of mice treated with SCV-07 prior to receiving a low-dose aerosol of virulent M. tuberculosis.

### DETAILED DESCRIPTION OF THE PREFERRED EMBODIMENTS

The present invention is applicable to animals capable of forming antibodies in an immune reaction, such as mammals including humans, livestock and pets, as well as birds such as domesticated fowl.

As animals age, their immune response is reduced, and vaccination effectiveness diminished due to the prevalance of low affinity antibody response. Accordingly, the invention is particularly applicable for use with humans over the age of 45, particularly those of age 50 and above.

The invention is also applicable to persons whose immune systems are compromised due to illness, surgery or medication, such as transplant patients who are immunodeficient as a result of administration of anti-rejection drugs such as cyclosporin.

It is believed that the present invention is applicable to all vaccines, and vaccine types, including killed or inactivated virus, DNA vaccines, peptide subunit vaccines and recombinant vaccines. Examples of suitable vaccines include Influenza vaccine, Hemophilus influenzae vaccine, Hepatitis A virus vaccine, Hepatitis B virus vaccine, Hepatitis C virus vaccine, Tuberculosis vaccine, Herpes-Zoster virus vaccine, Cytomegalovirus vaccine, Pneumococcal pneumonia vaccine, Meningococcal meningitis vaccine, Diphtheria vaccine, Tetanus vaccine, Rabies vaccine, Helicobacter pylori vaccine, polio vaccine and smallpox vaccine.

In one preferred embodiment the present invention provides a pharmaceutical composition for enhancing effectiveness of a Tuberculosis vaccine, such as the Calmette and Guérin Bacillus (BCG) vaccine. BCG is used in many countries with a high prevalence of TB to prevent childhood tuberculosis, meningitis and miliary disease. While BCG vaccine may protect children from contracting tuberculosis, it provides variable efficacy in adults. In this embodiment a tuberculosis vaccine such as BCG is administered together with the immunomodulator SCV-07 to provide enhanced vaccine efficacy in both adults and children.

It also is believed that the invention is applicable to any future vaccine, such as a vaccine which may be developed for vaccination against the AIDS virus, SARS or the avian influenza virus.

Generally, vaccines are administered in amounts within the range of from about 1×10⁻⁹ g to about 1×10⁻³ g, and more typically within the range from about 1×10⁻⁸ g to about 1×10⁻⁴ g.

Vaccine effectiveness-enhancing amounts of the immunomodulator compound of Formula A generally are administered in amounts within the range of about 0.001-1000 ug/kg body weight of the recipient, preferably in amounts of about 0.1-100 μg/kg, more preferably about 0.3-30 μg/kg, and most preferably about 10 μg/kg.

Immunomodulator compounds in accordance with the present invention, comprise immunomodulator compound of Formula A:

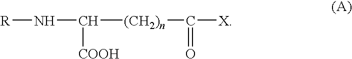

In Formula A, n is 1 or 2, R is hydrogen, acyl, alkyl or a peptide fragment, and X is an aromatic or heterocyclic amino acid or a derivative thereof. Preferably, X is L-tryptophan or D-tryptophan.

Preferred derivatives of the aromatic or heterocyclic amino acids for “X” are: amides, mono- or di-(C₁-C₆) alkyl substituted amides, arylamides, and (C₁-C₆) alkyl or aryl esters. Preferred acyl or alkyl moieties for “R” are: branched or unbranched alkyl groups of 1 to about 6 carbons, acyl groups from 2 to about 10 carbon atoms, and blocking groups such as carbobenzyloxy and t-butyloxycarbonyl. Preferably the carbon of the CH group shown in Formula A has a stereoconfiguration, when n is 2, that is different from the stereoconfiguration of X.

Preferred embodiments utilize compounds such as γ-D-glutamyl-L-tryptophan, γ-L-glutamyl-L-tryptophan, γ-L-glutamyl-Nᵢₙ-formyl-L-tryptophan, N-methyl-γ-L-glutamyl-L-tryptophan, N-acetyl-γ-L-glutamyl-L-tryptophan, γ-L-glutamyl-D-tryptophan, β-L-aspartyl-L-tryptophan, and β-D-aspartyl-L-tryptophan. Particularly preferred embodiments utilize γ-D-glutamyl-L-tryptophan, sometimes referred to as SCV-07. These compounds, methods for preparing these compounds, pharmaceutically acceptable salts of these compounds and pharmaceutical formulations thereof are disclosed in U.S. Pat. No. 5,916,878, incorporated herein by reference.

In accordance with one aspect of the present invention, the immunomodulator compound of Formula A may be administered before and/or concurrently with administration of the vaccine.

The present invention may be particularly effective when administered in connection with a secondary (booster) vaccination dose. Secondary or booster vaccination doses typically are administered within a time period of beginning at the time the first (primary) dose of the vaccine is administered to about 2 months after administration of the first vaccine dose, preferably within about 0-45 days of the first vaccine dose, more preferably within about 10-30 days of administration of the first vaccine dose, and according to some embodiments within about 10-20 days of administration of the first vaccine dose.

In accordance with one embodiment, a dose of the immunomodulator compound of Formula A is administered to a recipient several days prior to administration of a secondary (booster) vaccine dose, most preferably about 3-4 days prior to administration of the secondary (booster) vaccine dose. In another preferred embodiment, an immunomodulator compound also is administered concurrently with administration of the secondary (booster) vaccine dose. In a particularly preferred embodiment, an immunomodulator compound is administered concurrently with administration of the first vaccine dose and with administration of the secondary (booster) vaccine dose. In an additional preferred embodiment the immunomodulator may be administered with administration of the first vaccine dose and with each subsequent vaccine booster dose that is administered.

Administration of the immunomodulator compound of Formula A and vaccine may take place by any suitable means, such as injection, infusion or orally. It has been found that compounds of Formula A are effective when administered orally as peroral dosage forms.

Pharmaceutical compositions containing the immunomodulators of the present invention or their pharmaceutically acceptable salts may be prepared according to conventional pharmaceutical compounding techniques. See, for example, Remington's Pharmaceutical Sciences, 18th Ed. (1990, Mack Publishing Co., Easton, Pa.). Typically, an immunomodulating amount of the active ingredient will be admixed with a pharmaceutically acceptable carrier to form a composition suitable for administration per os.

For oral administration, the compounds may be formulated into solid or liquid preparations such as capsules, pills, tablets, lozenges, melts, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent may be encapsulated to make it stable for passage through the gastrointestinal tract.

The pharmaceutically acceptable carrier may include non-toxic, inert solid, semi-solid liquid fillers, diluents, encapsulating material, formulation auxiliary of any type, or simply a sterile aqueous medium, such as saline. Examples of materials that may serve as pharmaceutically acceptable carriers include sugars, such as lactose, glucose and sucrose; starches such as corn starch and potato starch; cellulose and its derivatives such as sodium carboxymethyl cellulose, ethyl cellulose and cellulose acetate; magnesium stearate; powdered tragacanth; malt; gelatin; talc; excipients such as cocoa butter and suppository waxes; oils; such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate, agar; buffering agents such as magnesium hydroxide and aluminum hydroxide; alginic acid collidons, such as collidon 30 and collidon CL; pyrogen-free water; isotonic saline, Ringer's solution; ethyl alcohol and phosphate buffer solutions, as well as other non-toxic compatible substances used in pharmaceutical formulations.

Wetting agents, emulsifiers and lubricants such as sodium lauryl sulfate and magnesium stearate, as well as coloring agents, releasing agents, coating agents, sweetening, flavoring and perfuming agents, preservatives and antioxidants may also be present in the composition, according to the judgment of the formulator. Examples of pharmaceutically acceptable antioxidants include, but are not limited to, water soluble antioxidants such as ascorbic acid, cysteine hydrochloride, sodium bisulfite, sodium metabisulfite, sodium sulfite, and the like; oil soluble antioxidants, such as ascorbyl palmitate, butylated hydroxyanisole (BHA), butylated hydroxytoluene (BHT), lecithin, propyl gallate, aloha-tocopherol and the like; and the metal chelating agents such as citric acid, ethylenediamine tetraacetic acid (EDTA), sorbitol, tartaric acid, phosphoric acid and the like.

For oral administration, the compounds may be formulated into solid or liquid preparations such as capsules, pills, tablets, lozenges, melts, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent may be encapsulated to make it stable for passage through the gastrointestinal tract.

Effective amounts of Formula A compound may be determined by routine dose-titration experiments. Exact individual dosages, as well as daily dosages of the pharmaceutical composition may be determined according to standard medical principles under the direction of a physician or veterinarian for use in humans or animals. In one aspect the pharmaceutical composition may be used to enhance the production of antibodies in an animal in response to administration of decreased quantities of vaccine.

When a vaccine and the immunomodulator compound of Formula A are administered concurrently, they may be provided as a single composition including the vaccine and the immunomodulator compound of Formula A.

Compositions including a vaccine and/or the immunomodulator compound of Formula A may also include one or more pharmaceutically acceptable carriers and optionally other therapeutic ingredients. Formulations suitable for injection or infusion include aqueous and non-aqueous sterile injection solutions which may optionally contain antioxidants, buffers, bacteriostats and solutes which render the formulations isotonic with the blood of the intended recipient, and aqueous and non-aqueous sterile suspensions which may include suspending agents and thickening agents. The formulations may be presented in unit-dose or multi-dose containers, for example, sealed ampoules and vials, and may be stored in a freeze-dried (lyophilized) condition requiring only the addition of the sterile liquid carrier, for example, water for injection, immediately prior to use.

Administration of SCV-07 produces significant immunomodulatory effects, including dose-dependent changes in the subpopulation composition of thymic cells. The percentage of immature CD4⁻CD8⁻ and CD4⁺ cells increases, while the percentage of double positive CD4⁺ CD8⁺ cells decreases. Changes in cytokine production which occur following administration of SCV-07 are consistent with an increase in Th1 helper T cell subsets. The intimate involvement of Th1 CD4 cells in supporting antibody responses and the effect of SCV-07 on Th1 cells are consistent with the properties of the compound as a vaccine response enhancer. The largest effect of SCV-07 administration is seen at a dose of 1 μg/kg for per os administration and at dose of 0.1-1.0 ug/kg for i/p administration.

Changes in cytokine production following administration of SCV-07 suggest that the immunomodulatory effects of SCV-07 administered either i/p or per os are mediated by influencing events in Th1-dependent immune responses. It has been found that SCV-07 produces essentially identical effects when administered either orally or intraparenterally. Thus, administration of the compound can produce immunomodulatory effects when administered in a vaccine-effectiveness-enhancing amount to enhance production and affinity of antibodies in an animal in response to a vaccine administered either in an initial or a subsequent booster dose.

### Example 1

### Evaluation of the Efficacy of Oral versus Parenteral Administration of SCV-07 for Augmenting Immune Response in Mice

Materials and Methods: Eighteen (18) to twenty (20) gram (g) male mice, purchased from the Rappolovo Animal Facility, Russia, were used in the studies described below. C57BL/6 (B6) mice were used to evaluate the effect of SCV-07 in non-immunized mice. CBAxC57BL/6 (F1) mice were used for studies of the effects of SCV-07 on the antigen-specific immune response. Mice were kept under the standard animal facility conditions and were provided with a standard diet and water ad libitum. Each treatment group included 4-5 mice.

To evaluate the oral administration of SCV-07, tablets composed of 0.1 mg SCV-07; 2.0 mg magnesium stearate; 6.0 mg collidon 30; 10.0 mg collidon CL; and sufficient D-mannitol were prepared. Tablets of approximately 205 mg SCV-07 were formulated such that they readily dissolved in the stomach of test animals.

A 205 mg tablet having 0.1 mg SCV-07 per tablet was crushed and suspended in 200 ul of distilled water and administered to mice at does of 0.01, 1.0, 10.0 and 100.0 μg of SCV-07 per kg of body mass. A suspension containing filling agents alone, which corresponded to the maximum amount of filling agents in the doses of SCV-07 administered to experimental animals, was used a control.

Immunization with ovalbumin (OvA): To study antigen-specific immune response to SCV-07, mice were immunized with 5 μg/mouse of OvA obtained from Sigma. The OvA was administered subcutaneously to the crest in a volume of 200 μl. Mice were immunized on day 1 with OvA in complete Freund's adjuvant (CFA; Sigma) and on day 15 with the antigen in incomplete Freund's adjuvant (IFA; Sigma). The immune response was analyzed on day 29. Mice were also immunized with 10 μg/mouse of OvA using Alum as an adjuvant.

SCV-07 was administered peroral to the groups of mice at doses from 0.01 μg per kg to 100 μg per kg per os through an esophageal catheter on days 1 to 3 following administration of OvA and again on days 15 to 17. Other groups received SCV-07 by intraperitoneal injection (i/p) at doses of either 0.1 or 1.0 μg/kg of body mass, administered at the same times following immunization with QvA. In addition, groups of animals were immunized with OvA in FA as above, but only received one round of three SCV-07 treatments (either on days 1-3, days 15-17, or days 24-26) to evaluate the effects of a single treatment.

Isolation of thymocytes and splenocytes: Mouse thymuses and spleens were removed from treated animals under aseptic conditions 72 hours after the last administration of SCV-07. Thymuses and spleens from mice of each treatment group were pooled, and then minced with scissors. The minced tissues were suspended in sterile PBS to form cell suspensions, filtered through a double gauze layer and centrifuged. Cells were washed twice with PBS, re-suspended in RPMI-1640 (Sigma) with 2.0 mM of L-glutamine, 50 μM of mercaptoethanol, and 10 μg/ml of gentamycin (complete medium); and cell counts were made. (The splenocyte suspension was washed with a 0.86% solution of NH₄Cl to pre-lyse erthrocytes.)

Thigh bones were removed from treated mice under aseptic conditions, and the medullary cavities were lanced and washed with PBS. Fragments of the bone marrow were minced by passing the tissue through a syringe needle. The resulting cell suspension was filtered through a double gauze layer and washed in PBS. After erythrocytes were lysed, the cells were counted and transferred to flat bottom culture plates. Due to an almost complete lack of spontaneous proliferation, the absolute values of proliferation, which were expressed as counts per minute (CPM), were determined and the Con A-induced proliferation of thymic cells from animals of different groups were compared.

Assessment of Mitogen- and Antigen-Induced Proliferation: Thymocytes (1×10⁶/well) and splenocytes (5×10⁵/well) in 96-well flat-bottom culture plates (Costar, Cambridge, Mass.) and stimulated with 0.5 or 1.0 μg/ml of ConA (Sigma) in complete medium supplemented with either 10% or 2% fetal calf serum (FCS), respectively, and incubated in a CO₂-incubator for 72 hours at a temperature of 37° C., under absolute humidity. Antigen-specific proliferation was studied by stimulating thymocytes and splenoctyes in vitro with different concentrations of OvA for 96 h.

Twenty (20) to twenty-four (24) hours prior to completion of incubation, 3H-labeled thymidine (5 μCi/ml) was added to the cells. The cells were then transferred to fiberglass filters with a Titertek® semi-automatic cell harvester. Thymidine uptake was measured using a RackBeta 1217 (Wallac) liquid scintillation β-counter. For quantitative analysis of proliferation, the stimulation index (SI) was calculated as a ratio of the intensity of label inclusion in the stimulated cells to that in non-stimulated cells.

Assessment of Mitogen- and Antigen-Induced Production of Cytokines: Thymic cells (1×10⁶/well) in complete medium with 2% FCS were stimulated with ConA (0, 1, and 2 μg/ml). Splenocytes (1×10⁶/well) in complete medium with 5% FCS were stimulated with ConA (0, 1, and 5 μg/ml) and with OvA (0-100 μg/ml). Supernatants from ConA-stimulated cultures were collected after 24 hours. Supernatants from OvA-stimulated cultures were collected after 72 hours. Supernatants from both cultures were frozen, and kept at 20° C. Concentrations of IL-2, IL-4, IL-10, IL-12R40 and IFNγ in the supernatants were determined by solid-phase EIA with EIA Expansion Kits, available from BD Biosciences (formerly Pharmingen), in accordance with the manufacturer's instructions. In addition, biological activity of IL-2 in the supernatants was estimated in a bioassay using the IL-2-dependent CTLL-2 cell line.

Analysis by Flow Cytometry: Suspensions of thymocytes and splenocytes were washed twice with PBS/1% FCS, incubated with monoclonal antibodies direct to the specific antigens indicated in Table 1 below at concentrations recommended by the manufacturer. The cells were incubated with the antibodies for 20 min. at 4° C., washed 2 times with PBS/1% FCS, and analyzed with the EPICS® XL™ flow cytometer (Beckman Coulter) with the gate set to analyze mononuclear cells using side and forward scatter plots.

TABLE 1

| | | |
|---|---|---|
| mouse anti-mouse CD90.1 | Thy1 | Caltag |
| rat anti-mouse CD8a-PE | clone 5H10 | Caltag |
| rat anti-mouse CD25-PE | clone PC61 5.3 | Caltag |
| rat anti-mouse CD4-FITC | clone CT-CD4 | Caltag |
| hamster anti-mouse CD3-FITC | clone 500A2 | Caltag |
| hamster anti-mouse CD69-FITC | clone H1.2F3 | Pharmingen |
| FITC-conjugated hamster IgG | isotype control | Caltag |
| FITC-conjugated rat IgG2a | isotype control | Caltag |
| PE-conjugated rat IgG2b | isotype control | Caltag |
| PE-conjugated rat IgG1 | isotype control | Caltag |

Statistical analysis: Statistical analyses were performed with Microsoft Office Excel 2003 (Microsoft Corp.) and SPSS v.12.0 for Windows (SPSS Inc.). To calculate the confidence of differences in thymocyte proliferation, the paired Student test with unequal deviation was used. The confidence of anti-OvA antibody titers differences was determined with non-parametric Mann-Whitney test for two samples. The differences were considered to be significant if p<0.05.

Immunomodulatory Activity of SCV-07 After Oral Administration: Thy1-positive lymphocytes, the precursors of T-cells in the bone marrow, increased in a dose-dependent manner in mice after treatment with SCV-07 per os. These increases were analogous to those observed in mice which received SCV-07 i.p (Table 2).

TABLE 2
Influence of SCV-07 on expression of Thy1 marker
by bone marrow cells

| | Route of | |
|---|---|---|
| SCV-07 μg/kg | administration | Thy-1 positive cells, % |
| 0 | i/p | 11.5 ± 1.6 |
| 1.0 | i/p | 15.4 ± 0.1 |
| 0.1 | per os | 12.9 ± 1.2 |
| 1.0 | per os | 14.9 ± 1.3 |
| 10.0 | per os | 16.0 ± 0.7 |
| 100.0 | per os | 16.0 ± 1.2 |

The expression of T-cell subpopulation markers (CD3, CD4, and CD8) and activation markers (CD69 and CD25) on thymic cells and splenocytes was studied ex vivo 72 hours after the last treatment with SCV-07. No significant differences in the subpopulation composition of T-cells or in expression of their activation markers were revealed in mice which received SCV-07 i/p or per os (data not shown). However, on subsequent cultivation for 48 hours without stimulation, the number of thymic cells which expressed CD69 and CD25 markers significantly increased. The expression of the CD69 marker increased after oral administration of SCV-07 tablets at doses of 0.1 and 1.0 μg/kg of body mass as well as after i/p administration at a dose of 1.0 μg/kg. The number of cells which expressed CD25 markers increased in mice that received SCV-07 either per os at all the doses or i/p at a dose of 1.0 μg/kg of body mass (Table 3).

TABLE 3
Influence of SCV-07 on expression of activation markers
on thymic cells cultured in vitro

| SCV-07 | Route of | | |
|---|---|---|---|
| μg/kg | administration | CD69-positive, % | CD25-positive cells, % |
| 0 | i/p | 3.28 | 0.38 |
| 0.1 | i/p | 2.03 | 0.30 |
| 1.0 | i/p | 7.86 | 1.72 |
| Filling | Tablet, per os | 2.46 | 0.28 |
| agents | | | |
| 0.1 | Tablet, per os | 3.35 | 0.7 |
| 1.0 | Tablet, per os | 4.61 | 0.79 |
| 10.0 | Tablet, per os | 2.24 | 0.71 |

Evaluation of the proliferation of thymic cells stimulated by different doses of ConA demonstrated that in mice which received SCV-07 either per Os or i/p, proliferation of thymic cells stimulated by a suboptimal dose of ConA (1 μg/ml) significantly increased (Table 4) while proliferation of cells stimulated by the optimal dose of ConA did not significantly change (data not shown). The minimal SCV-07 dose (0.1 μg/kg) which caused significant increase in proliferation of thymic cells after oral administration was the same as that for i/p administration.

TABLE 4
Influence of SCV-07 on Proliferation of Thymic Cells
Stimulated by ConA (1 μg/ml)

| | | Spontaneous | Induced | |
|---|---|---|---|---|
| | | proliferation | proliferation | |
| SCV-07 | Route of | (cpm) | (cpm) | Stimulation |
| μg/kg | administration | M ± SD | M ± SD | index |
| 0 | — | 265 ± 23 | 6314 ± 432 | 23.8 |
| 0.1 | i/p | 268 ± 114 | 14261 ± 1400*** | 53.3 |
| 1.0 | i/p | 342 ± 83 | 20995 ± 6425* | 61.4 |
| 0.01 | per os | 238 ± 64 | 6698 ± 1465 | 28.1 |
| 0.1 | per os | 250 ± 75 | 9754 ± 1633* | 39.1 |
| 1.0 | per os | 198 ± 44 | 20658 ± 5873* | 104.6 |
| 10.0 | per os | 246 ± 51 | 13905 ± 2443** | 56.6 |
| 100.0 | per os | 272 ± 98 | 27589 ± 1331*** | 101.4 |

*p < 0.05 as compared to control;
**p < 0.01 as compared to control;
***p < 0.001 as compared to control

The influence of SCV-07 tablets on thymic cell proliferation was compared to the effect of tablet filing agents alone. As evident from Table 5, SCV-07 at doses of 0.1 to 10 μg/kg significantly augmented proliferation of cells stimulated by a suboptimal dose of ConA, as compared with filling agents at the maximum concentration. The minimal dose of SCV-07 given as a crushed tablet which caused significant enhancement of proliferation (Table 5; 0.1 μg/kg) was the same as that for SCV-07 without filling agents (Table 4).

TABLE 5
Influence of SCV-07 on proliferation of thymic cells
stimulated by ConA (1 μg/ml)

| | | Spontaneous | Induced | |
|---|---|---|---|---|
| | | proliferation | proliferation | Stim- |
| SCV-07 | Route of | (cpm) | (cpm) | ulation |
| μg/kg | administration | M ± SD | M ± SD | index |
| 0 | i/p | 494 ± 58 | 6354 ± 1426 | 12.9 |
| 0.1 | i/p | 251 ± 36 | 9897 ± 678^{#} | 39.4 |
| 1.0 | i/p | 555 ± 20 | 33418 ± 3490^{##} | 49.7 |
| Filling | Tablet, per os | 220 ± 41 | 3842 ± 705 | 17.5 |
| agents | | | | |
| 0.01 | Tablet, per os | 308 ± 104 | 3144 ± 582 | 10.2 |
| 0.1 | Tablet, per os | 395 ± 136 | 9900 ± 2021** | 25.1 |
| 1.0 | Tablet, per os | 355 ± 62 | 10248 ± 1913* | 28.9 |
| 10.0 | Tablet, per os | 317 ± 77 | 7675 ± 1456* | 24.2 |

*p < 0.05 as compared to control (filling agent - per os)
**p < 0.01 as compared to control (filling agent - per os)
^{#}p < 0.05 as compared to control (i/p)
^{##}p < 0.01 as compared to control (i/p)

Considering that significant enhancement of proliferation by SCV-07 might be due to its influence on IL-2 production, IL-2 activity was studied with the IL-2-dependent CTLL-2 line. It was demonstrated that SCV-07 received either per os or i/p at doses of 0.1 and 1.0 μg/kg significantly enhanced IL-2 production by thymic cells stimulated by ConA (Table 6).

TABLE 6
Influence of SCV-07 on IL-2 production by
ConA-stimulated thymic cells

| | | |
|---|---|---|
| SCV-07 | Route of | IL-2 production (U/ml), M ± SD |

| μg/kg | administration | ConA 1 μg/ml | ConA 2 μg/ml |
|---|---|---|---|
| 0 | — | 0.00 ± 0.00 | 0.42 ± 0.16 |
| 0.1 | i/p | 0.25 ± 0.08 | 1.40 ± 0.38* |
| 1.0 | i/p | 0.92 ± 0.06* | 1.60 ± 0.15* |
| 0.01 | per os | 0.00 ± 0.00 | 0.44 ± 0.11 |
| 0.1 | per os | 0.10 ± 0.13 | 1.00 ± 0.13* |
| 1.0 | per os | 1.06 ± 0.37 | 3.31 ± 0.01* |
| 10.0 | per os | 0.25 ± 0.12 | 0.39 ± 0.12 |
| 100.0 | per os | 0.31 ± 0.15 | 1.19 ± 0.30 |

*p < 0.05 when compared to PBS-treated group

IL-2 production by splenocytes stimulated by ConA, especially at the suboptimal dose of 1 μg/ml, also was significantly enhanced in mice which received SCV-07 per os at doses of 0.1-100.0 μg/kg and was slightly higher after i/p injection of SCV-07 at doses of 0.1 and 1.0 μg/kg (Table 7).

TABLE 7
Influence of SCV-07 on IL-2 production by
ConA-stimulated splenocytes

| | | IL-2 production (U/ml), M ± SD | | |
|---|---|---|---|---|
| SCV-07 | Route of | | | ConA |
| μg/kg | administration | Spontaneous | ConA 1 μg/ml | 2 μg/ml |
| 0 | — | 0.6 ± 0.3 | 4.5 ± 0.3 | 50.2 ± 0.1 |
| 0.1 | i/p | 2.0 ± 0.3 | 16.1 ± 0.8* | 93.4 ± 28.3 |
| 1.0 | i/p | 2.7 ± 0.3 | 15.6 ± 0.3** | 99.3 ± 6.1* |
| 0.01 | per os | 0.0 ± 0.0 | 10.4 ± 1.1 | 66.9 ± 12.6 |
| 0.1 | per os | 0.0 ± 0.0 | 7.5 ± 0.1* | 85.8 ± 6.6 |
| 1.0 | per os | 0.3 ± 0.5 | 10.0 ± 0.4** | 60.8 ± 1.2* |
| 10.0 | per os | 0.0 ± 0.0 | 9.1 ± 1.3 | 56.4 ± 2.1 |
| 100.0 | per os | 0.3 ± 0.5 | 14.9 ± 2.4 | 79.3 ± 16.3 |

*p < 0.05 when compared to PBS-treated group
**p < 0.01 when compared to PBS-treated group

In all animal groups which received SCV-07 per os or i/p, the IFN-γ and IL-4 content was enhanced in supernatants of splenocytes stimulated by a suboptimal dose of ConA. However, if splenocytes were stimulated by a higher dose of ConA (5 μg/ml), IFN-γ production was enhanced only after i/p treatment with SCV-07, and production of IL-4 in cell supernatants of all mice treated with SCV-07 was lower than the control group (Table 8). Splenocytes stimulated by a high dose of ConA (with decreased IL-4 production) also had a tendency for a decrease in IFN-γ after SCV-07 per os. After i/p treatment with SCV-07, the level of IFN-γ increased, while that of IL-4 was significantly reduced, compared to that in control.

TABLE 8
Influence of SCV-07 on IFN-γ and IL-4 production by
ConA-stimulated splenocytes

| | | IFN-γ (pg/ml) | | | IL-4 (pg/ml) | | |
|---|---|---|---|---|---|---|---|
| SCV-07 | Route of | | ConA | ConA | | ConA | ConA |
| μg/kg | administration | Spontaneous | 1 μg/ml | 5 μg/ml | Spontaneous | 1 μg/ml | 5 μg/ml |
| 0 | — | 1368 | 6344 | 84358 | 34.7 | 40.4 | 312.3 |
| 0.1 | i/p | 1393 | 13089 | 87051 | 4.2 | 62.1 | 277.5 |
| 1.0 | i/p | 1738 | 30951 | 142155 | 0.0 | 52.8 | 152.9 |
| 0.01 | per os | 0 | 15499 | 63227 | 6.1 | 98.7 | 284.9 |
| 0.1 | per os | 0 | 15695 | 65692 | 0.0 | 78.9 | 229.6 |
| 1.0 | per os | 1629 | 11411 | 47683 | 25.3 | 95.8 | 260.0 |
| 10.0 | per os | 0 | 8238 | 66185 | 19.4 | 53.8 | 275.0 |
| 100.0 | per os | 0 | 12990 | 43670 | 27.1 | 83.1 | 192.0 |

Immunomodulatory activity of SCV-07 in animals pre-immunized with ovalbumin: F1 mice were immunized with ovalbumin, either with Freund's adjuvant (FA), which is known to stimulate a cell-mediated response (Th1 T-cell polarization), or with Alum adjuvant (Alum), which is known to stimulate the antibody response (Th2 T-cell polarization). Results of the studies of mice immunized with 5 μg of OvA with FA, and treated with SCV-07 per os or i/p, are shown in Tables 8-16.

SCV-07, administered either per os or i/p, produced no significant effect on OvA-specific proliferation of splenocytes (Table 9), although the stimulation indices of animal groups which received SCV-07 were slightly lower than those in control groups after incubation with OvA at 50 μg/ml.

TABLE 9
Influence of SCV-07 on OvA-induced proliferation of splenocytes

| | SCV- | | Proliferation of splenocytes | | | | |
|---|---|---|---|---|---|---|---|
| | 07 | Route of | Spontaneous | OvA 10 μg/ml | | OvA 50 μg/ml | |
| Immunization | μg/kg | admin. | cpm, M ± SD | cpm, M ± SD | SI | Cpm, M ± SD | SI |
| PBS | 0 | i/p | 1583 ± 716 | 1674 ± 598 | 1.06 | 3188 ± 604 | 2.01 |
| OvA/FA | 0 | i/p | 6492 ± 1410 | 11207 ± 1895 | 1.73 | 11345 ± 1618 | 1.75 |
| OvA/FA | 0.1 | i/p | 10726 ± 1947 | 11683 ± 2285 | 1.09 | 14195 ± 3304 | 1.32 |
| OvA/FA | 1.0 | i/P | 9760 ± 1650 | 10260 ± 2808 | 1.05 | 16313 ± 1057 | 1.67 |
| OvA/FA | 0.1 | per os | 8786 ± 1259 | 11304 ± 1317 | 1.29 | 11923 ± 1623 | 1.36 |
| OvA/FA | 1.0 | per os | 7013 ± 1698 | 8864 ± 2675 | 1.26 | 10457 ± 1740 | 1.49 |
| OvA/FA | 10.0 | per os | 10665 ± 887 | 13585 ± 1102 | 1.27 | 12903 ± 881 | 1.21 |

Levels of IL-2 production in splenocytes stimulated by OvA are shown in Tables 10 and 11. As evident from Table 11 (data are shown as percent, compared to spontaneous production), in splenocytes of mice which received SCV-07 at a dose of 0.1 μg/kg i/p or per os, IL-2 production was significantly reduced. In animals which received SCV-07 at a dose of 1.0 μg/kg i/p and of 1.0 μg/kg and 10 μg/kg per os, the level of IL-2 production increased compared to that of control after stimulation by low doses of antigen, and yet decreased after stimulation by the higher doses of antigen.

TABLE 10
Influence of SCV-07 on OvA-stimulated production of
IL-2 by splenocytes

| | | | Production of IL-2 by splenocytes, | | | | |
|---|---|---|---|---|---|---|---|
| | | | pg/ml | | | | |
| | SCV-07 | Route of | Stimulation in vitro by OvA, μg/ml | | | | |
| Immunization | μg/kg | admin. | 0 | 1 | 10 | 25 | 100 |
| PBS | 0 | i/p | 81.4 | 68.5 | 83.9 | 80.2 | 89.5 |
| OvA/FA | 0 | i/p | 125.6 | 51.4 | 217 | 442.2 | 404 |
| OvA/FA | 0.1 | i/p | 78.4 | 10.2 | 72.7 | 85.8 | 95.5 |
| OvA/FA | 1.0 | i/p | 147.7 | 116.5 | 317.3 | 363.1 | 564.4 |
| OvA/FA | 0.1 | per os | 211.8 | 68.7 | 92.9 | 252.5 | 94.8 |
| OvA/FA | 1.0 | per os | 122.4 | 82.7 | 304.2 | 152.9 | 282.8 |
| OvA/FA | 10.0 | per os | 163 | 80.2 | 305.4 | 287.2 | 478.2 |

TABLE 11
Influence of SCV-07 on OvA-stimulated production
of IL-2 by splenocytes (% of control)

| | | | Production of IL-2 by | | | | |
|---|---|---|---|---|---|---|---|
| | | | splenocytes, % | | | | |
| | SCV-07 | Route of | Stimulation in vitro by OvA, μg/ml | | | | |
| Immunization | μg/kg | admin. | 0 | 1 | 10 | 25 | 100 |
| PBS | 0 | i/p | 100 | 84.2 | 103.1 | 98.5 | 110.0 |
| OvA/FA | 0 | i/p | 100 | 40.9 | 172.8 | 352.1 | 321.7 |
| OvA/FA | 0.1 | i/p | 100 | 13.0 | 92.7 | 109.4 | 121.8 |
| OvA/FA | 1.0 | i/p | 100 | 78.9 | 214.8 | 245.8 | 382.1 |
| OvA/FA | 0.1 | per os | 100 | 32.4 | 43.9 | 119.2 | 44.8 |
| OvA/FA | 1.0 | per os | 100 | 67.6 | 248.5 | 124.9 | 231.0 |
| OvA/FA | 10.0 | per os | 100 | 49.2 | 187.4 | 176.2 | 293.4 |

IFN-γ levels in supernatants of in vitro antigen-stimulated splenocytes from mice immunized with OvA/FA are shown in Tables 12 and 13. The spontaneous production of IFN-γ significantly increased in control as compared to that in non-immunized animals. In all groups which received SCV-07, spontaneous production of IFN-γ increased even more significantly (Table 11). At the same time, OvA-stimulated production of IFN-γ decreased in SCV-07 treated animals compared to control.

TABLE 12
Influence of SCV-07 on OvA-stimulated IFN-γ production by splenocytes

| | | | Production of IFN-γ by splenocytes, % | | | | |
|---|---|---|---|---|---|---|---|
| | | Route of | Stimulation in vitro by OvA, μg/ml | | | | |
| Immunization | SCV-07 μg/kg | admin. | 0 | 10 | 25 | 50 | 100 |
| PBS | 0 | i/p | 1090 | 1305 | 1563 | 2090 | 3424 |
| OvA/FA | 0 | i/p | 4477 | 6705 | 5761 | 6216 | 6074 |
| OvA/FA | 0.1 | i/p | 8442 | 10691 | 7542 | 9959 | 10490 |
| OvA/FA | 1.0 | i/p | 8191 | 11386 | 9194 | 8110 | 7340 |
| OvA/FA | 0.1 | per os | 7784 | 9292 | 9427 | 11056 | 10548 |
| OvA/FA | 1.0 | per os | 7693 | 9860 | 6720 | 8884 | 8683 |
| OvA/FA | 10.0 | per os | 7870 | 6586 | 5209 | 5971 | 5888 |

TABLE 13
Influence of SCV-07 on OvA-stimulated IFN-γ production
by splenocytes (% of control)

| | | | Production of IFN-γ by | | | | |
|---|---|---|---|---|---|---|---|
| | | | splenocytes, % | | | | |
| | SCV-07 | Route of | Stimulation in vitro by OvA, μg/ml | | | | |
| Immunization | μg/kg | admin. | 0 | 10 | 25 | 50 | 100 |
| PBS | 0 | i/p | 100 | 119.7 | 143.4 | 191.8 | 314.1 |
| OvA/FA | 0 | i/p | 100 | 149.8 | 128.7 | 138.8 | 135.7 |
| OvA/FA | 0.1 | i/p | 100 | 126.6 | 89.3 | 118.0 | 124.3 |
| OvA/FA | 1.0 | i/p | 100 | 139.0 | 112.2 | 99.0 | 89.6 |
| OvA/FA | 0.1 | per os | 100 | 119.4 | 121.1 | 142.0 | 135.5 |
| OvA/FA | 1.0 | per os | 100 | 128.2 | 87.3 | 115.5 | 112.9 |
| OvA/FA | 10.0 | per os | 100 | 83.7 | 66.2 | 75.9 | 74.8 |

In all animals, the levels of IL-4 in supernatants of OvA-stimulated splenocytes were below the sensitivity of the test-system used (6 pg/ml). The levels of IL-10 in OvA stimulated splenocytes of mice immunized with OvA/FA are shown in Tables 14 and 16. In animals which received SCV-07 i/p at a dose of 0.1 μg/kg, a relative decrease of OvA-stimulated IL-10 content was observed, while in mice which received SCV-07 per os the levels of IL-10 moderately increased.

TABLE 14
Influence of SCV-07 on OvA-stimulated IL-10 production by splenocytes

| | | | Production of IL-10 by splenocytes, pg/ml | | | | |
|---|---|---|---|---|---|---|---|
| | | Route of | Stimulation in vitro by OvA, μg/ml | | | | |
| Immunization | SCV-07 μg/kg | admin. | 0 | 10 | 25 | 50 | 100 |
| PBS | 0 | i/p | 102.2 | 129.8 | 199 | 210.9 | 306.8 |
| OvA/FA | 0 | i/p | 424.2 | 643.9 | 732.6 | 783.7 | 879.9 |
| OvA/FA | 0.1 | i/p | 785.1 | 989.4 | 1234.3 | 1194.2 | 1502.4 |
| OvA/FA | 1.0 | i/p | 457.4 | 813.4 | 774.1 | 852.4 | 963.8 |
| OvA/FA | 0.1 | per os | 702.5 | 958.9 | 1478.2 | 1739.2 | 1895.4 |
| OvA/FA | 1.0 | per os | 407.6 | 953.1 | 984.1 | 1120.7 | 1292.2 |
| OvA/FA | 10.0 | per os | 627.3 | 831.6 | 1092.7 | 1356.1 | 1569.3 |

TABLE 15
Influence of SCV-07 on OvA-stimulated IL-10
production by splenocytes (% of control)

| | | | Production of IL-10 by | | | | |
|---|---|---|---|---|---|---|---|
| | | | splenocytes, % | | | | |
| | SCV-07 | Route of | Stimulation in vitro by OvA, μg/ml | | | | |
| Immunization | μg/kg | admin. | 0 | 10 | 25 | 50 | 100 |
| PBS | 0 | i/p | 100 | 127.0 | 194.7 | 206.4 | 300.2 |
| OvA/FA | 0 | i/p | 100 | 151.8 | 172.7 | 184.7 | 207.4 |
| OvA/FA | 0.1 | i/p | 100 | 126.0 | 157.2 | 152.1 | 191.4 |
| OvA/FA | 1.0 | i/p | 100 | 177.8 | 169.2 | 186.4 | 210.7 |
| OvA/FA | 0.1 | per os | 100 | 136.5 | 210.4 | 247.6 | 269.8 |
| OvA/FA | 1.0 | per os | 100 | 233.8 | 241.4 | 275.0 | 317.0 |
| OvA/FA | 10.0 | per os | 100 | 132.6 | 174.2 | 216.2 | 250.2 |

Levels of IL-12r40 in supernatants of OvA-stimulated splenocytes are shown in Tables 16 and 17. In the control group, a decrease in antigen-specific IL-12R40 production was found compared to that in non-immunized animals. In animal groups which received SCV-07 i/p (at both doses) and in groups which received SCV-07 per os at a dose of 10.0 μg/kg, there was less of a decrease.

TABLE 16
Influence of SCV-07 on OvA-stimulated production of IL-12R40 by splenocytes

| | | | Production of IL-12R40 by splenocytes, pg/ml | | | | |
|---|---|---|---|---|---|---|---|
| | SCV-07 | Route of | Stimulation in vitro by OvA, μg/ml | | | | |
| Immunization | μg/kg | admin. | 0 | 10 | 25 | 50 | 100 |
| PBS | 0 | i/p | 1288.6 | 1366.8 | 1278.5 | 1162.9 | 1079.1 |
| OvA/FA | 0 | i/p | 1508.3 | 500.4 | 424.5 | 401.8 | 460.9 |
| OvA/FA | 0.1 | i/p | 1285.7 | 492.4 | 499.6 | 420.8 | 522.8 |
| OvA/FA | 1.0 | i/p | 1524.0 | 933.8 | 739.7 | 630.3 | 616.4 |
| OvA/FA | 0.1 | per os | 1307.6 | 478.1 | 317.4 | 267.0 | 246.5 |
| OvA/FA | 1.0 | per os | 1362.3 | 366.3 | 324.5 | 360.8 | 342.4 |
| OvA/FA | 10.0 | per os | 1455.1 | 769.3 | 592 | 471.9 | 464.8 |

TABLE 17
Influence of SCV-07 on OvA-Stimulated IL-12r40
Production by Splenocytes (% of control)

| | | | Production of IL-12R40 by | | | | |
|---|---|---|---|---|---|---|---|
| | SCV- | | splenocytes, % | | | | |
| | 07 | Route of | Stimulation in vitro by OvA, μg/ml | | | | |
| Immunization | μg/kg | admin | 0 | 10 | 25 | 50 | 100 |
| PBS | 0 | i/p | 100 | 106.1 | 99.2 | 90.2 | 83.7 |
| OvA/FA | 0 | i/p | 100 | 33.2 | 28.1 | 26.6 | 30.6 |
| OvA/FA | 0.1 | i/p | 100 | 38.3 | 38.9 | 32.7 | 40.7 |
| OvA/FA | 1.0 | i/p | 100 | 61.3 | 48.5 | 41.4 | 40.4 |
| OvA/FA | 0.1 | per os | 100 | 36.6 | 24.3 | 20.4 | 18.9 |
| OvA/FA | 1.0 | per os | 100 | 26.9 | 23.8 | 26.5 | 25.1 |
| OvA/FA | 10.0 | per os | 100 | 52.9 | 40.7 | 32.4 | 31.9 |

Results of the study of mice immunized with 10 μg of OvA with Alum adjuvant and treated with SCV-07 per os or i/p, are shown in Tables 18-23. No significant differences in proliferation of splenocytes were seen in mice of the control group compared to those which received SCV-07 (data not shown).

Antigen-specific production of IL-2 by splenocytes is shown in Tables 18 and 19. IL-2 production in OvA-stimulated splenocytes increased in all groups which received SCV-07, compared to control. The IL-2 production was the highest in mice which received SCV-07 at a dose of 1.0 μg/kg (i/p) and of 10.0 μg/kg (per os), Table 18.

TABLE 18
Influence of SCV-07 on OvA-stimulated production
of IL-2 by splenocytes

| | | | Production of IL-2 by | | | | |
|---|---|---|---|---|---|---|---|
| | SCV- | | splenocytes, pg/ml | | | | |
| | 07 | Route of | Stimulation in vitro by OvA, μg/ml | | | | |
| Immunization | μg/kg | admin | 0 | 1 | 10 | 25 | 100 |
| PBS | 0 | i/p | 124.0 | 243.7 | 103.5 | 99.3 | 92.8 |
| OvA/Alum | 0 | i/p | 303.1 | 246.6 | 109.8 | 122.9 | 209.2 |
| OvA/Alum | 0.1 | i/p | 163.0 | 190.3 | 87.5 | 83.5 | 84.8 |
| OvA/Alum | 1.0 | i/p | 113.4 | 173.7 | 167.9 | 353.8 | 205.8 |
| OvA/Alum | 0.1 | per os | 172.9 | 163.0 | 124.5 | 255.5 | 90.8 |
| OvA/Alum | 1.0 | per os | 160.9 | 92.8 | 181.9 | 121.3 | 120.2 |
| OvA/Alum | 10.0 | per os | 289.4 | 551.9 | 252.5 | 218.7 | 300.8 |

TABLE 19
Influence of SCV-07 on OvA-stimulated production
of IL-2 by splenocytes (% of control)

| | | | Production of IL-2 by | | | | |
|---|---|---|---|---|---|---|---|
| | | | splenocytes, % | | | | |
| | SCV-07 | Route of | Stimulation in vitro by OvA, μg/ml | | | | |
| Immunization | μg/kg | admin | 0 | 1 | 10 | 25 | 100 |
| PBS | 0 | i/p | 100 | 196.5 | 83.5 | 80.1 | 74.8 |
| OvA/Alum | 0 | i/p | 100 | 81.4 | 36.2 | 40.5 | 69.0 |
| OvA/Alum | 0.1 | i/p | 100 | 116.7 | 53.7 | 51.2 | 52.0 |
| OvA/Alum | 1.0 | i/p | 100 | 153.2 | 148.1 | 312.0 | 181.5 |
| OvA/Alum | 0.1 | per os | 100 | 94.3 | 72.0 | 147.8 | 52.5 |
| OvA/Alum | 1.0 | per os | 100 | 57.7 | 113.1 | 75.4 | 74.7 |
| OvA/Alum | 10.0 | per os | 100 | 190.7 | 87.2 | 75.6 | 103.9 |

A significant increase of OvA-specific production of IFN-γ by splenocytes was observed only in mice which received SCV-07 per os at a dose of 0.1 μg/kg. In other animal groups, production of IFN-γ did not differ compared to that in control (Tables 20 and 21).

TABLE 20
Influence of SCV-07 on OvA-stimulated production
of IFN-γ by splenocytes

| | | | Production of IFN-γ by | | | | |
|---|---|---|---|---|---|---|---|
| | | | splenocytes, pg/ml | | | | |
| | SCV-07 | Route of | Stimulation in vitro by OvA, μg/ml | | | | |
| Immunization | μg/kg | admin. | 0 | 10 | 25 | 50 | 100 |
| PBS | 0 | i/p | 8580 | 8729 | 7471 | 7471 | 8406 |
| OvA/Alum | 0 | i/p | 4932 | 4975 | 5844 | 5844 | 5228 |
| OvA/Alum | 0.1 | i/p | 9365 | 8816 | 8028 | 8028 | 8048 |
| OvA/Alum | 1.0 | i/p | 7129 | 9986 | 7982 | 7982 | 8155 |
| OvA/Alum | 0.1 | per os | 4642 | 5775 | 6969 | 6969 | 7310 |
| OvA/Alum | 1.0 | per os | 5620 | 5523 | 5795 | 5795 | 6860 |
| OvA/Alum | 10.0 | per os | 4266 | 2045 | 5066 | 5066 | 5976 |

TABLE 21
Influence of SCV-07 on OvA-stimulated production of IFN-γ
by splenocytes (% of control)

| | | | Production of IFN-γ by | | | | |
|---|---|---|---|---|---|---|---|
| | | | splenocytes, % | | | | |
| | SCV-07 | Route of | Stimulation in vitro by OvA, μg/ml | | | | |
| Immunization | μg/kg | admin | 0 | 10 | 25 | 50 | 100 |
| PBS | 0 | i/p | 100 | 101.7 | 87.1 | 87.1 | 98.0 |
| OvA/Alum | 0 | i/p | 100 | 100.9 | 118.5 | 118.5 | 106.0 |
| OvA/Alum | 0.1 | i/p | 100 | 94.1 | 85.7 | 85.7 | 85.9 |
| OvA/Alum | 1.0 | i/p | 100 | 140.1 | 112.0 | 112.0 | 114.4 |
| OvA/Alum | 0.1 | per os | 100 | 124.4 | 150.1 | 150.1 | 157.5 |
| OvA/Alum | 1.0 | per os | 100 | 98.3 | 103.1 | 103.1 | 122.1 |
| OvA/Alum | 10.0 | per os | 100 | 47.9 | 118.8 | 118.8 | 140.1 |

Similar to experiments with the use of FA, in all animal groups the levels of IL-4 production in supernatants of OvA-stimulated splenocytes were below the sensitivity of the test system.

The antigen-specific production of IL-10 by splenocytes from mice which received SCV-07 was significantly lower than that of control. This effect was the strongest in animal groups which received SCV-07 i/p (doses of 0.1 and 1.0 μg/kg), and was slightly less pronounced in groups which received SCV-07 per os at the same doses (Table 22).

TABLE 22
Influence of SCV-07 on OvA-stimulated production of IL-10 by splenocytes

| | | | Production of IL-10 by splenocytes, pg/ml | | | | |
|---|---|---|---|---|---|---|---|
| | SCV-07 | Route of | Stimulation in vitro by OvA, μg/ml | | | | |
| Immunization | μg/kg | admin | 0 | 10 | 25 | 50 | 100 |
| PBS | 0 | i/p | 1042.7 | 1403 | 1432.2 | 1515.6 | 1266.5 |
| OvA/Alum | 0 | i/p | 784.8 | 1942.4 | 1780.1 | 1815.5 | 2268.6 |
| OvA/Alum | 0.1 | i/p | 2177.8 | 1762.5 | 1946.1 | 1851.3 | 2189.5 |
| OvA/Alum | 1.0 | i/p | 2035.7 | 1758.9 | 1829.8 | 1584.7 | 1840.7 |
| OvA/Alum | 0.1 | per os | 1351.1 | 1587.9 | 1419.5 | 1783.6 | 1741.5 |
| OvA/Alum | 1.0 | per os | 1741.5 | 2220.9 | 1957.2 | 1920.4 | 1898.4 |
| OvA/Alum | 10.0 | per os | 816.4 | 1127.5 | 1272.5 | 1608.4 | 1780.1 |

TABLE 23
Influence of SCV-07 on OvA-stimulated production of IL-10
by splenocytes (% of control)

| | | | Production of IL-10 by | | | | |
|---|---|---|---|---|---|---|---|
| | | | splenocytes, % | | | | |
| | SCV-07 | Route of | Stimulation in vitro by OvA, μg/ml | | | | |
| Immunization | μg/kg | admin. | 0 | 10 | 25 | 50 | 100 |
| PBS | 0 | i/p | 100 | 134.6 | 137.4 | 145.4 | 121.5 |
| OvA/Alum | 0 | i/p | 100 | 247.5 | 226.8 | 231.3 | 289.1 |
| OvA/Alum | 0.1 | i/p | 100 | 80.9 | 89.4 | 85.0 | 100.5 |
| OvA/Alum | 1.0 | i/p | 100 | 86.4 | 89.9 | 77.8 | 90.4 |
| OvA/Alum | 0.1 | per os | 100 | 117.5 | 105.1 | 132.0 | 128.9 |
| OvA/Alum | 1.0 | per os | 100 | 127.5 | 112.4 | 110.3 | 109.0 |
| OvA/Alum | 10.0 | per os | 100 | 138.1 | 155.9 | 197.0 | 218.0 |

No significant differences in OvA-induced production of IL-12r40 by splenocytes were observed in control versus mice which received SCV-07 either per os or i/p (data not shown).

### Example 2

### Evaluation of the Ability of SCV-07 to Enhance BCG-Induced Protection in a Murine Model of Pulmonary Tuberculosis

The dipeptide SCV-07 (gamma-D-glutamyl-L-tryptophan) was evaluated in a murine pulmonary tuberculosis model and found to enhance the protective effect of M. bovis Bacillus Calmette-Guerin (BCG) when administered as a vaccine.

Mice of about 20-25 grams in weight were allowed to acclimate for three weeks prior to initiation of the study. The mice were divided into sixteen treatment groups containing five (5) mice each. SCV-07 was administered as a 0.1 ml injection into the right flank of each treated animal. The treatment groups received either 0.01 μg, 0.2 μg or 2.0 μg of the dipeptide, which corresponds to doses of 0.4 μg/kg, 8 μg/kg and 80 μg/kg. Groups 2 to 15 were vaccinated subcutaneously with 0.1 ml (10⁵ colony forming units (CFUs)) of BCG vaccine, which was administered into the left flank of the animal on day 5 of the study.

Control groups 1, 2, 15 and 16 received no SCV-07. Groups 1 and 16 also received no BCG vaccination. Group 15 was vaccinated with BCG on day 5 and received daily injections of saline.

Treatment groups 3, 7 and 11 received daily injections of the immunomodulator SCV-07 on days 1 to 5; groups 4, 8, and 12 received daily injections on days 1 to 30 and groups 5, 9, and 13 received daily injections on days 1 to 5 and on day 7, day 11, day 14, day 18, day 21, day 25 and day 28. Groups 6, 10 and 14 received daily injections of SCV-07 on days 6 to 30.

Groups 3, 4, 5, and 6 received 0.01 μg of SCV-07 a day according to the temporal administration schedule indicated above. Groups 7, 8, 9, and 10 received 0.2 μg and Groups 11, 12, 13 and 14 received 2.0 μg.

The treatment regimens used in the study are summarized below in Table 24.

TABLE 24
Experimental Protocol

| | | | DAYS |
|---|---|---|---|
| GROUP | VACCINE | IMMUNOMODULATOR | ADMINISTRATED |
| 1 | — | | |
| 2 | BCG | — | — |
| 3 | BCG | 0.01 μg SCV-07 | D1-D5 |
| 4 | BCG | 0.01 μg SCV-07 | D1-D30 |
| 5 | BCG | 0.01 μg SCV-07 | D1-5, D7, 11, 14, |
| | | | 18, 21, 25, 28 |
| 6 | BCG | 0.01 μg SCV-07 | D6-D30 |
| 7 | BCG | 0.20 μg SCV-07 | D1-D5 |
| 8 | BCG | 0.20 μg SCV-07 | D1-D30 |
| 9 | BCG | 0.20 μg SCV-07 | D1-5, D7, 11, 14, |
| | | | 18, 21, 25, 28 |
| 10 | BCG | 0.20 μg SCV-07 | D6-D30 |
| 11 | BCG | 2.0 μg SCV-07 | D1-D5 |
| 12 | BCG | 2.0 μg SCV-07 | D1-D30 |
| 13 | BCG | 2.0 μg SCV-07 | D1-5, D7, 11, 14, |
| | | | 18, 21, 25, 28 |
| 14 | BCG | 2.0 μg SCV-07 | D6-D30 |
| 15 | BCG | saline | D1-D30 |
| 16 | — | | — |

On day 31 all mice were exposed to a low-dose aerosol of virulent M. tuberculosis strain H₃₇Rv. On the following day the animals of group 16 were sacrificed and their lung tissue harvested and plated on nutrient 7H11 agar to determine the number of colony forming units of M. tuberculosis in the initial challenge with antigen.

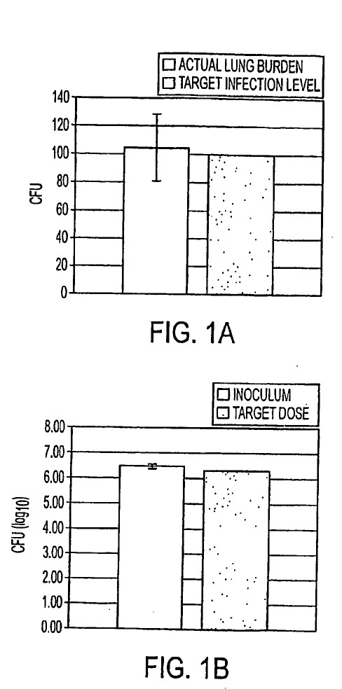

Bacterial colonies were counted three weeks after plating. FIG. 1A provides the mean CFU (+/−1 standard deviation) per total lung tissue received by control animals compared to the target dose of 100 CFU per lung, which was the desired dose for the murine pulmonary tuberculosis model. These results indicate that the desired low-dose aerosol infection in the murine model of pulmonary tuberculosis was achieved. FIG. 1B provides an analysis of aliquots of the actual inoculum used for the aerosol infection which indicates that the aliquots contained an infective dose of 2×10⁶ CFU/ml. Data are expressed as mean CFU per plate (n=4).

On day 61, thirty days following infection, all rice in groups 1 to 15 were euthanized by CO₂ inhalation. The lungs and spleen of each mouse were removed. The right lung of each mouse was placed into a polypropylene tube and stored frozen at −80° C. The left lung was infused with five (5) ml of 10% neutral-buffered formalin (NBF) and placed in 5 ml of 10% NBF with the spleen from the same animal. On day 66 the right lung was homogenized in five (5) ml of sterile phosphate-buffered saline and subsequently plated onto nutrient 7H11 agar to determine the lung bacterial burden of M. tuberculosis for each mouse. The lung tissue homogenate was serially diluted, plated onto 7H11 agar and incubated at 37° C. for approximately three weeks. Individual colonies of BCG were counted. The results are presented in Table 25 below, expressed as log₁₀ CFU per milliliter (ml) of homogenate.

TABLE 25
CFUs of BCG per ml of Lung Homogenate in Treated Mice

| GROUPS | M1 | M2 | M3 | M4 | M5 | MEAN | MEDIAN |
|---|---|---|---|---|---|---|---|
| 1 | 6.6 | 6.6 | 5.8 | 5.4 | 5.9 | 6.1 | 5.9 |
| 2 | 5.5 | 5.5 | 5.2 | 5.2 | 5.2 | 5.3 | 5.2 |
| 3 | 4.8 | 5.3 | 4.9 | 5.3 | 4.8 | 5.0 | 4.9 |
| 4 | 5.1 | 4.8 | | 5.0 | 4.3 | 4.8 | 4.9 |
| 5 | 5.1 | 5.0 | 4.6 | 5.1 | 4.8 | 4.9 | 5.0 |
| 6 | 5.4 | 5.5 | 5.5 | 5.7 | | 5.5 | 5.5 |
| 7 | 4.8 | 4.8 | 5.0 | 5.1 | 5.0 | 4.9 | 5.0 |
| 8 | 4.5 | 4.7 | 4.6 | 4.7 | | 4.6 | 4.6 |
| 9 | 4.6 | 4.4 | 4.9 | 5.1 | 4.8 | 4.8 | 4.8 |
| 10 | | 5.4 | 5.2 | | | 5.3 | 5.3 |
| 11 | 5.1 | 4.8 | 5.1 | 4.8 | 5.1 | 5.0 | 5.1 |
| 12 | 5.1 | 4.8 | 5.3 | 5.3 | 4.6 | 5.0 | 5.1 |
| 13 | 4.8 | 4.8 | 4.7 | 5.1 | 5.3 | 4.9 | 4.8 |
| 14 | 5.3 | | 5.4 | 5.3 | 5.5 | 5.4 | 5.4 |
| 15 | 5.5 | 5.7 | 5.6 | 5.9 | 5.8 | 5.7 | 5.7 |

Empty cells indicate mice for which CFU counts were not obtained due to contaminated lung homogenates.

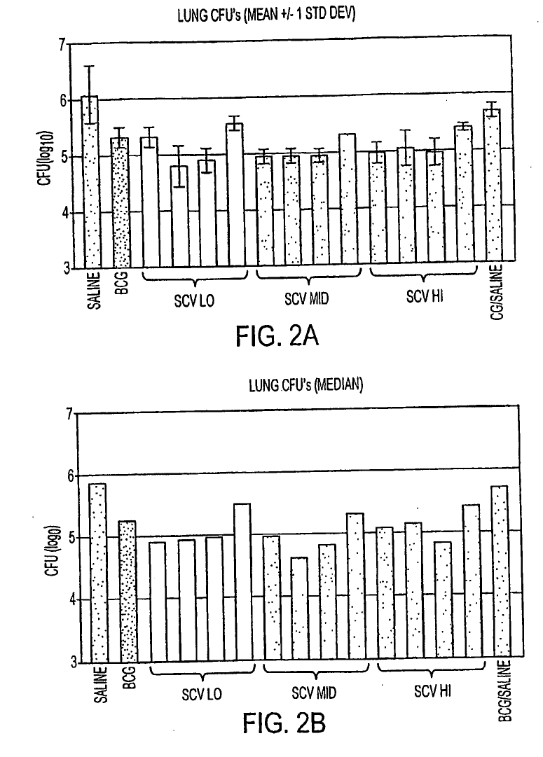

As demonstrated by the results above, mice that received SCV-07 therapy prior to BCG vaccination had an enhanced immunological response against a subsequent challenge with a low dose of virulent M. tuberculosis. The optimal enhancing effect of SCV-07 for vaccination with BCG is seen with the intermediate 0.20 μg dose of the immunomodulator, which is about 8-10 μg/kg of SCV-07. These results are also presented graphically in FIG. 2, which shows the mean (+/−1 standard deviation) lung colony forming units in panel A and the median CFUs in panel B.

This study confirms the ability of SCV-07 to enhance the protective effect of pre-inoculation vaccination with M. bovis BCG in a murine model of pulmonary tuberculosis. The study measured the effect of three different doses of the dipeptide, given according to four different administration schedules relative to the administration of BCG inoculation. The schedules included early administration, in which SCV-07 was administered before BCG vaccination (days 1 to 5); early/late administration, in which SCV-07 was administered both before and after BCG vaccination (days 1 to 30); early/late intermittent administration, in which SCV-07 was administered before BDG vaccination (days 1 to 5) and continued intermittently after vaccination (days 7, 11, 14, 18, 21, 25 and 28) and late administration, in which SCV-07 was administered only after BCG vaccination. As shown in Table 25 and in FIG. 2, enhancement of the protective effect provided by BCG vaccination, was better when SCV-07 was administered prior to BCG vaccination. The 0.20 μg dose (approximately 8-10 μg/kg of SCV-07) provided the best enhancement of the BCG protective effect in the murine model.

### Example 3

### Influence of the Immunomodulator SCV-07 on the Effectiveness of Vaccination Against Viral Hepatitis B

The dipeptide SCV-07 (gamma-D-glutamyl-L-tryptophan) was evaluated in human subjects with the spontaneous form of secondary immunodeficiency and found to enhance the protective effect of vaccination against hepatitis B. The studies were the first use of SCV-07 as an immunomodulator for enhancement of vaccine effectiveness.

The protocol of the placebo-controlled, randomized study of the efficacy of vaccination against viral hepatitis B was approved by the committee of ethics of the Chelyabinsk State Medical Academy. Participants in the study had a spontaneous form of secondary immunodeficiency with clinical manifestations of infectious syndrome. Diagnosis of the infectious syndrome was made in accordance with the standards of diagnostics and treatment of secondary immunodeficiencies recommended by the institute of Immunology of the Ministry of Public Health of the RF, Moscow. (R. M. Kahaitov, N. I. Iljina, L. V. and Luss et al., 2002) All subjects gave their personal consent to participate in the study and submitted a signed informed consent form.

Criteria for participation in the study included the presence of one or more symptoms of infectious syndrome for one or more years and the absence of severe somatic pathology. Participants who had an acute disease or aggravated chronic disease, who were pregnant or nursing; who had used immuno-modulators or vaccines during a six month period prior to the study; who had been vaccinated against hepatitis, had a preceding case of hepatitis, or had hepatitis B markers in their blood; or who were participating in another study were excluded.

Study participants were students of the Chelyabinsk State Medical Academy, who were due to receive obligatory vaccination against viral hepatitis B as a priority group according to Order No. 226/79 dated Jun. 3, 1996, of the Ministry of Medical Industry of Russia and the State Committee for Sanitary Epidemiological Inspection of Russia. The study, including participant examinations, treatment and vaccination, was conducted at the medical training institution of Chelyabinsk State Clinical Hospital No. 2, the Clinic and the Research Institute of Immunology of the Chelyabinsk State Medical Academy. The participant group included sixty-nine (69) subjects between the ages of nineteen (19) and twenty-five (25). Eighteen (18) of the participants were male and fifty-one (51) were female.

The participants were divided into three groups, thirty-seven (37) subjects with infectious syndrome were immunized with the vaccine against hepatitis B without administration of the immunomodulator SCV-07 (Group I), thirty-two (32) subjects with infectious syndrome received SCV-07 and the vaccine (Group II) and a control treatment group that did not have infectious syndrome (Group III). This group, which received SCV-07 and hepatitis B vaccine, included thirty-three (33) essentially healthy students from the Chelyabinsk State Medical Academy.

The hepatitis vaccine Engerix-B® produced by SmithKlineBeecham-Biomed (Russia) was administered as a course of 3 doses given at months 0, 1, and 6. Immunological examinations of the subjects were carried out before the first administration of the vaccine and at one month after completion of the course of immunization. All participants were seen by an allergologist-immunologist once during the first three months following vaccine administration. Subjects receiving SCV-07 were examined daily by the allergologist-immunologist. If needed, immunocompromised subjects were also seen by doctors of different medical specialties, including gynecologists, gastroenterologists, ear nose and throat (ENT) specialists, infection specialists, dermatologists, surgeons, etc.

Data on the incidence of episodes and relapses of chronic diseases for each immunocompromised subject were recorded during pre- and post vaccination periods and used to compare the immunological clinical parameters of each subject in the pre- and post vaccination periods. Chronic diseases that were tracked included diseases of the ears, nose and throat, urinary systems, genital systems and gastrointestinal tract, and diseases of the skin, hair and nails (the integumentary tissues).

Participants were evaluated to determine levels of leukocytes in peripheral blood, leukocytary formula, the presence of marker specific lymphocytes (CD3, CD4, CD8, CD16, CD95 and CD20), lysosomal activity of neutrophils; oxygen-dependent metabolism with spontaneous and induced hematocrit (HCT) tests, phagocytary function of neutrophils according to the latex particle absorption model; immunoglobulin IgA, IgM, IgG concentrations; total hemolytic activity of complement (CH50) in blood serum and for circulating immune complexes. Titers of antibodies to HBAg in the blood serum were determined by immunoenzyme analysis in paired sera using the test system of Vector-Best, Ltd., Novosibirsk, Russia. A concentration of 10 MU/liter (l) was assumed to be a protective hepatitis B titer.

Participants in the study had the following clinical manifestations of infectious syndrome: sixty-seven percent (67%) had acute respiratory viral infections (ARVI) more than four times a year; forty-nine percent (49%) had chronic inflammatory diseases of the ears, nose and throat; forty-five percent (45%) suffered from recurrent herpes viral infections; thirty percent (30%) had chronic inflammatory diseases of the gastrointestinal tract; thirty-three (33%) had recurrent infections of the skin and subcutaneous adipose cellular tissues. Study participants with the spontaneous form of secondary immunodeficiency typically had more than one chronic inflammatory disease. Seventy-one percent (71%) of the subjects with infectious syndrome had three or more different clinical manifestations of a chronic inflammatory disease. Subjects with a single clinical manifestation made up only three percent (3%) of Groups I and II. Approximately fifty-nine percent (58.8%) of the participants had ARVI 5-9 times a year; 24.7% had ARVI 1 to 4 times a year and the remaining 13.4% of the immunodeficiency subjects had an ARVI less than once a year.

Approximately thirty percent (30%) of the study participants had symptoms of secondary immunodeficiency throughout their lives with a typical wave-form manifestation of symptoms where periods of relative wellness alternated with periods of frequent occurrence of disease. Approximately eleven percent (11.3%) of participants developed symptoms for five to ten years prior to the start of the study. Approximately thirty-eight percent (38.1%) of the participants developed symptoms during the 3 to 5 year period preceding the study. Approximately twenty-one percent (20.6%) of the study participants were diagnosed with secondary immunodeficiencies one to three years before initiation of the study.

The immunomodulator SCV-07 was administered at the same time as the hepatitis B vaccine to determine if SCV-07 increased the efficacy of vaccination and to determine if it would correct the immunity disorders of subjects with infectious syndrome. A one milligram (1 mg) dose of SCV-07 was administered as an intra muscular injection to the 32 subjects of Group 2 of the study on days one to five, where day one was the day the first dose of the hepatitis B vaccine was administered. One (1) mg of SCV-07 was also administered with the second and third doses of vaccine one month and 6 months following administration of the initial vaccine dose. Study participants in Group I received injections of physiological saline which were administered on the same schedule as the SCV-07 to provide a placebo control group.

The clinical manifestations of infectious syndrome changed considerably in vaccinated participants that also received SCV-07. The incidence of ARVI was sharply reduced in those subjects who previously had reported ARVI between 5 and 9 times a year. Incidence of ARVI was also reduced in those participants who had reported ARVI at a frequency of 1 to 4 times a year. The number of subjects who reported no occurrences of ARVI in the year following vaccination increased. Thirty-five percent (35%) of subjects who previously had aggravated chronic diseases of the ears, nose and throat three to four times a year had no aggravated incidents during the first year after vaccination.

The number of participants who experienced episodes of herpes infections or outbreaks did not change substantially following administration of SCV-07. However, there was a redistribution of episodes of infections or outbreaks. Before vaccination and treatment with SCV-07, 40% of the subjects suffered from herpes infections five times a year or more. After vaccination and SCV-07 administration no participants reported outbreaks of this frequency. However, the number of participants reporting outbreaks one to two times during a year increased from thirteen (13%) to fifty-three (53%). Thirty-four percent of participants who were vaccinated and received SCV-07 did not have episodes or outbreaks of herpes.

The frequency of chronic diseases of the gastrointestinal tract and the urogenital system did not change among vaccinated participants with infectious syndrome that received SCV-07.

The effect of SCV-07 on the immune status of subjects with spontaneous form of secondary immunodeficiency. The immune status of subjects with infectious syndrome that received SCV-07 at the time of hepatitis B vaccination was determined immediately before vaccination and at one month after the final dose of vaccine was administered (seven months after the initial dose was administered) with a series of clinical assays. The results of these assays allowed an assessment of the effects of vaccination in the presence of the immunomodulator on the immune status of the study participants. The experimental protocol allowed comparisons of date collected from participants who received SCV-07 with data from participants who received only the hepatitis vaccine and with untreated subjects. Data from the participants of Group II were also compared before and after vaccination.

TABLE 26
Concentration of leukocytes and lymphocytes in
peripheral blood before and after vaccination

| | | Studied groups | | | |
|---|---|---|---|---|---|
| | | | | Patients | Healthy |
| | | Patients of group 2 | | of group | subjects |
| | | Before | After | After | After |
| Studied | Statistical | vaccination, | vaccination | vaccination | vaccination |
| Indices | Indices | n = 21 | n = 21 | n = 15 | n = 22 |
| Leukocytes × | M + m p | 5.455 ± 0.214 | 5.938 ± 0.351 | 5.08 ± 0.34 | 6.04 ± 0.31 |
| 10⁹/l | | n = 22 | p1 > 0.05 | p2 > 0.05 | p3 > 0.05 |
| Lymphocytes % | M + m p | 22.00 ± 1.86 | 27.81 ± 2.04 | 24.07 ± 2.43 | 19.05 ± 1.13 |
| | | n = 22 | p1 > 0.05 | p2 > 0.05 | p3 < 0.05 |
| Lymphocytes × | M + m p | 1.440 ± 0.145 | 1.608 ± 0.096 | 1.179 ± 0.128 | 1.19 ± 0.11 |
| 10⁹/l | | n = 22 | p1 < 0.05 | p2 < 0.05 | p3 < 0.05 |

Note for Tables 26-30:
p1 - statistical confidence of differences in the group of subjects treated with SCV-07 before and after vaccination;
p2 - statistical confidence of differences between the group of subjects treated with SCV-07 and the group of untreated subjects after vaccination;
p3 - statistical confidence of differences between the group of subjects treated with SCV-07 and the group of healthy individuals after vaccination.

Determinations of the concentration of subpopulations of lymphocytes in the peripheral blood of participants with infectious syndrome after vaccination against heptatitis B indicated that in subjects who also received SCV-07, the population of CD3+ lymphocytes increases such that the level of CD3+ cells approaches the population of CD3+ cells seen in healthy participants after vaccination. In addition, there also appears to be an increase in the numbers of CD4+ lymphocytes in peripheral blood of participants treated with sCV-07 when compared to levels present before vaccination. While this difference does not appear to be a statistically significant increase in the assays used for participants who received SCV-07 at the time of vaccination, it may nonetheless contribute to the clinical improvements observed in the Group II participants.

TABLE 27
Concentration of the subpopulations of
lymphocytes before and after vaccination

| | | Studied groups | | | |
|---|---|---|---|---|---|
| | | Patients of | | | |
| | | group 2 | | Patients of | Healthy of |
| | | Before | After | group 1 after | group 3 after |
| Studied | Statistical | vaccination | vaccination | vaccination n = | vaccination |
| indices | indices | n = 21 | n = 16 | 15 | n = 22 |
| CD3+ (%) | M + m p | 26.81 ± 1.41 | 29.60 ± 2.41 | 22.67 ± 1.90 | 33.10 ± 1.88 |
| | | | p1 > 0.05 | p2 < 0.05 | p3 > 0.05 |
| CD4+ (%) | M + m p | 19.23 ± 1.08 | 20.13 ± 1.65 | 18.33 ± 0.97 | 22.86 ± 1.94 |
| | | | p1 > 0.05 | p2 > 0.05 | p3 > 0.05 |
| CD8+ (%) | M + m p | 19.82 ± 1.28 | 18.27 ± 1.01 | 18.40 ± 2.07 | 19.71 ± 1.53 |
| | | | p1 > 0.05 | p2 > 0.05 | p3 > 0.05 |
| CD4+/CD8+ | M + m p | 1.050 ± 0.112 | 1.111 ± 0.086 | 1.135 ± 0.106 | 1.203 ± 0.094 |
| conv. units | | | p1 > 0.05 | p2 > 0.05 | p3 > 0.05 |
| CD16+ (%) | M + m p | 15.41 ± 1.45 | 13.07 ± 1.18 | 13.93 ± 1.19 | 15.19 ± 1.24 |
| | | | p1 > 0.05 | p2 > 0.05 | p3 > 0.05 |
| CD95+ (%) | M + m p | 13.18 ± 0.97 | 12.60 ± 1.18 | 16.60 ± 1.94 | 14.62 ± 1.60 |
| | | | p1 > 0.05 | p2 > 0.05 | p3 > 0.05 |

Table 28 presents data from quantitative and functional studies of neutrophils present in healthy subjects (Group 3) and in subjects with infectious syndrome vaccinated against hepatitis (Group 1) and subjects who received SCV-07 at the time of vaccination. Vaccination against hepatitis B with or without the administration of SCV-07 did not substantially affect the quantitative characteristics of neutrophils in patients with infectious syndrome. Vaccination and treatment with SCV-07 did however change the functional activity of neutrophils significantly. As seen from the data, there was a tendency for the intensity and activity of phagocytosis of neutrophils to rise. The phagocytary number in examined patients also rose.

The spontaneous activity and intensity of neutrophils in the hemocrit (HCT) reaction in immunodeficient subjects increased somewhat in response to vaccination against hepatitis B and treatment with SCV-07. The activities were significantly lower (p<0.05) than comparable results obtained for subjects with the infectious syndrome who received only hepatitis vaccine and for healthy patients who received the vaccine and the immunomodulator SCV-07.

The data from the induced HCT test provide a different pattern of results. Neutrophils from subjects who received SCV-07 showed higher, more intense activities in the induced HCT test than neutrophils from those same subjects prior to vaccination. In addition, activities of neutrophils in the induced HCT test after completion of vaccination in subjects who received SCV-07 were significantly higher than the activities of neutrophils from subjects who were vaccinated but did not receive the immunomodulator (p<0.05). The healthy subjects of Group 3 had significantly higher activities in the induced HCT test that vaccinated subjects with infectious syndrome who received SCV-07.

The functional reserve of cells is an essential index of the performance of phagocytes. Vaccination lowers this index in all test groups. However, the index was essentially the same for subjects with infectious syndrome who received SCV-07 and for healthy patients. In these studies the functional reserve for phagocytes in vaccinated subjects was significantly lower that the functional reserve of phagocytes in subjects with infectious syndrome treated with SCV-07 and in healthy patients.

TABLE 28
Concentration and functional activity of
neutrophils before and after vaccination

| | | Studied groups | | | |
|---|---|---|---|---|---|
| | | Group 2 | | Group 1 | Group 3 |
| | | Before | After | After | After |
| Studied | Statistical | vaccination | vaccination | vaccination | vaccination |
| indices | indices | n = 21 | n = 16 | n = 15 | n = 22 |
| Neutrophils % | M + m p | 63.27 ± 2.15 | 56.56 ± 2.07 | 63.00 ± 2.83 | 63.55 ± 1.88 |
| | | | p1 > 0.05 | p2 > 0.05 | p3 > 0.05 |
| Neutrophils × | M + m p | 4.036 ± 0.36 | 3.403 ± 0.27 | 3.217 ± 0.26 | 3.77 ± 0.21 |
| 10⁹/l | | | p1 > 0.05 | p2 > 0.05 | p3 > 0.05 |
| Activity of | M + m p | 34.23 ± 3.10 | 46.88 ± 4.92 | 65.93 ± 3.64 | 60.66 ± 3.72 |
| phagocytosis % | | | p1 > 0.05 | p2 < 0.05 | p3 < 0.05 |
| Intensity of | M + m p | 0.94 ± 0.163 | 1.44 ± 0.189 | 2.79 ± 0.45 | 2.38 ± 0.204 |
| phagocytosis, | | | p1 > 0.05 | p2 < 0.05 | p3 < 0.05 |
| conv. units | | | | | |
| Phagocytary | M + m p | 2.64 ± 0.205 | 2.92 ± 0.255 | 4.35 ± 0.4 | 3.676 ± 0.22 |
| number, conv. | | | p1 > 0.05 | p2 < 0.05 | p3 < 0.05 |
| units | | | | | |
| Spontaneous | M + m p | 28.36 ± 4.23 | 34.50 ± 5.62 | 37.73 ± 4.10 | 47.41 ± 4.35 |
| HCT-test, | | | p1 > 0.05 | p2 < 0.05 | p3 < 0.05 |
| activity, % | | | | | |
| Spontaneous | M + m p | 0.379 ± 0.07 | 0.50 ± 0.093 | 0.555 ± 0.07 | 0.699 ± 0.08 |
| HCT-test, | | | p1 > 0.05 | p2 < 0.05 | p3 < 0.05 |
| index, conv. | | | | | |
| Induced HCT- | M + m p | 33.43 ± 3.60 | 48.63 ± 4.62 | 32.07 ± 4.68 | 60.0 ± 3.98 |
| test, activity, % | | | p1 > 0.05 | p2 < 0.05 | p3 > 0.05 |
| Induced HCT- | M + m p | 0.526 ± 0.06 | 0.732 ± 0.08 | 0.463 ± 0.08 | 0.974 ± 0.08 |
| test, index, | | | p1 > 0.05 | p2 < 0.05 | p3 < 0.05 |
| conv. units | | | | | |
| Functional | M + m p | 2.411 ± 0.784 | 1.929 ± 0.225 | 0.92 ± 0.183 | 1.73 ± 0.181 |
| reserve, conv. | | | p1 > 0.0 | p2 < 0.05 | p3 > 0.05 |
| units | | | | | |
| Lysosomal | M + m p | 235.8 ± 23.7 | 198.7 ± 22.5 | 158.0 ± 16.4 | 267.7 ± 20.9 |
| activity, conv. | | | p1 > 0.05 | p2 > 0.05 | p3 < 0.05 |
| units | | | | | |

Studies of immune system components involved in humoral immunity in patients with secondary immunodeficiency disorders that result in infectious syndrome presented in Table 29 did not identify any substantial differences in patients before and after vaccination with concurrent administration of SCV-07. There was a tendency for concentrations of CD20+ lymphocytes and immunoglobulin of the Gig class to increase in the blood (p>0.05). However there was no significant difference in the data obtained for the concentration of CD20+ lymphocytes and IgA, IgM and IgG in the blood of patients with infectious syndrome who were treated with SCV-07 at the time of vaccination and the concentrations in patients who did not receive the immunomodulator and the healthy patients of Group III.

However administration of SCV-07 during vaccination against hepatitis produces changes in the immune system of patients characterized by a moderate rise in the concentration of CD3+, CD4+/CD8+ lymphocytes, a tendency towards reduction of spontaneous HCT, a stronger induced HCT reaction and a considerable increase in the reserve of functional neutrophils. Thus, the data show that administration of SCV-07 produces a positive immunomodulating effect in patients with the spontaneous form of secondary immunodeficiency.

TABLE 29
Indices of humoral immunity before and after vaccination

| | | Studied groups | | | |
|---|---|---|---|---|---|
| | | Patients | | Patients | Healthy of |
| | | of group | | of group | group 3 |
| | | Before | After | After | After |
| Studied | Statistical | vaccination | vaccination | vaccination | vaccination |
| indices | indices | n = 21 | n = 16 | n = 15 | n = 22 |
| CD20+ % | M + m p | 14.45 ± 0.88 | 15.93 ± 1.20 | 14.67 ± 1.51 | 17.29 ± 1.44 |
| | | | p1 > 0.05 | p2 > 0.05 | p3 > 0.05 |
| IgA | M + m p | 2.292 ± 0.176 | 1.821 ± 0.087 | 1.897 ± 0.116 | 1.96 ± 0.135 |
| (g/l) | | | p1 > 0.05 | p2 > 0.05 | p3 > 0.05 |
| IgM | M + m p | 1.440 ± 0.072 | 1.179 ± 0.089 | 1.229 ± 0.080 | 1.13 ± 0.06 |
| (g/l) | | | p1 > 0.05 | p2 > 0.05 | p3 > 0.05 |
| IgG | M + m p | 8.196 ± 0.387 | 8.931 ± 0.481 | 8.47 ± 0.64 | 8.88 ± 0.36 |
| (g/l) | | | p1 > 0.05 | p2 > 0.05 | p3 > 0.05 |
| CH50, | M + m p | 62.75 ± 2.40 | 64.53 ± 4.11 | 58.69 ± 2.64 | 59.88 ± 1.79 |
| conv. | | | p1 > 0.05 | p2 > 0.05 | p3 > 0.05 |
| units | | | | | |
| CIC, | M + m p | 65.95 ± 4.93 | 53.93 ± 8.03 | 64.50 ± 8.53 | 90.02 ± 13.1 |
| conv. | | | p1 > 0.05 | p2 > 0.05 | p3 < 0.05 |
| units | | | | | |

Assessment of the protective immunity against the hepatitis B virus. The effectiveness of vaccination against hepatitis B is assessed by the number of antibodies to HBsAg that an organism produces The defensive (protective) titer of the antibodies to hepatitis B is considered to be a level of antibodies no less than 10 MU per liter in blood serum. Antibody titers were determined in vaccinated patients one month after immunization using an immunoenzyme analysis provided by the test-system of Vector Best Ltd., Novosibirsk.

The results of the study show that the level of antibodies to HBsAg in the blood of subjects was lowest in the Group I patients vaccinated against hepatitis without SCV-07 immunotherapy. (Antibody titers of about 154.0 MU/L) The difference in antibody titers in the subjects of Group 1 were considerably lower than those of immunodeficient subjects who received SCV-07 and of healthy subjects. The concentrations of antibodies to HBsAg in the blood of immunodeficient patients treated with SCV-07 at the time of immunization and of healthy patients were approximately the same, 263.3 MU/L for immunodeficient patients who received SCV-07 at the time of vaccination and 253.6 MU/L for healthy subjects. Comparison of this index did not reveal any significant difference in Groups I, II and III.

The number of immunodeficient patients with protective titers of antibodies to HBsAg one month after vaccination against hepatitis in Group I, which did not receive SCV-07, was 29 or 78.4% of the group. The numbers of patients with protective immunity in the untreated group was significantly lower than the number of patients in Groups II and III. 96.9% of the immunodeficient subjects treated with SCV-07 seroconverted; 93.9% of the healthy subjects of Group II seroconverted.

TABLE 30
Frequency of seroconversions one month after the end of vaccination

| | Examined groups | | | | | |
|---|---|---|---|---|---|---|
| | Group 1, | | Group 2, | | Group 3, | |
| | untreated | | SCV-07 | | healthy | |
| | n = 37 | | n = 32 | | n = 33 | |
| Studied index | Abs | % | Abs | % | Abs | % |
| Protective titer | 29 | 78.4 | 31 | 96.9** | 31 | 93.9* |
| against HBsAg | | | | | | |
| (>10 mMU/mL) | | | | | | |
| Antibodies to | 8 | 21.6 | 1 | 3.1** | 2 | 6.1* |
| HBsAg not | | | | | | |
| detected | | | | | | |

Note:
*statistically valid differences between the group of patients vaccinated without treatment and the group of healthy vaccinated patients;
**statistically valid differences between the group of patients vaccinated without treatment and the group of patients treated with SCV-07; (Fisher's exact test is applied).

Conclusion: Vaccination of patients with altered reactivity of the immune system has been recognized as a medical problem for decades. Human immune activity in respect to individual vaccines is different. It depends on many factors, the specific genetic features of the organism being the main factor. Moreover, the intensity of immune response is affected by the features of the introduced antigen, and the phenotypic features of an individual acquired throughout life. Different types of immunity disorders are largely significant, specifically the states of immunodeficiency.

Vaccination is less effective in patients with the infectious syndrome when vaccinated against hepatitis B without any immunotherapy. Administration of SCV-07 makes vaccination against hepatitis B more effective in patients with the spontaneous form of secondary immunodeficiency, with clinical manifestations of the infectious syndrome that is evidenced by significant increase of the proportion of patients with the protective level of titers of antibodies to HBsAg. The data presented make it apparent that the immunomodulator SCV-07 stimulates production of antibodies against the hepatitis B virus in patients with the infectious syndrome providing a higher level of protection against infections compared with patients vaccinated without immunocorrection.

Clinical manifestations of the infectious syndrome are altered when vaccination against hepatitis B is accompanied by administration of SCV-07. The number of patients with chronic diseases of ENT organs, frequent ARVI aggravations and herpes infection relapses are significantly reduced. Administration of SCV-07 in these patients is accompanied by improved immunological responses. The achieved clinical effects of SCV-07 probably relate to its immunostimulating action.

The studies discussed above indicate that it is advisable to administer SCV-07 to patients with secondary immunodeficiency infectious syndrome concurrently with administration of a vaccine, in particular the Engerix-B vaccine for hepatitis. The immunomodulator SCV-07 is a safe adjuvant which reinforces vaccination against infectious agents and other antigens.

1. A pharmaceutical combination for enhancing vaccine effectiveness animals, comprising:
A) an immune response-triggering vaccine capable of stimulating production in an animal of antibodies to a disease-causing agent foreign to said animal; and
B) a vaccine effectiveness-enhancing amount of an immunomodulator compound of Formula A, which enhances production of said antibodies in said animal, in response to said vaccine;
C) wherein said vaccine and the immunomodulator compound of Formula A may be administered separately or together, and
D) wherein the immunomodulator compound of Formula A is 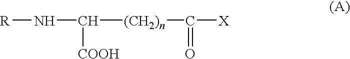
wherein n is 1 or 2, R is hydrogen, acyl, alkyl or a peptide fragment, and X is an aromatic or heterocyclic amino acid or a derivative thereof.

2. The pharmaceutical combination of claim 1, wherein the immunomodulator compound is SCV-07.

3. The pharmaceutical combination of claim 1, wherein said animal is human, and said vaccine is selected from the group consisting of Influenza vaccine, Hemophilus influenzae vaccine, Hepatitis A virus vaccine, Hepatitis B virus vaccine, Hepatitis C virus vaccine, Tuberculosis vaccine, Herpes-Zoster virus vaccine, Cytomegalovirus vaccine, Pneumococcal pneumonia vaccine, Meningococcal meningitis vaccine, Diphtheria vaccine, Tetanus vaccine, Rabies vaccine, Helicobacter pylori vaccine, polio vaccine and smallpox vaccine.

4. The pharmaceutical combination of claim 1, wherein said vaccine is in an amount of from about 1×10⁻⁹ g to about 1×10⁻³ g, and the immunomodulator compound of Formula A is in an amount of about 0.001-1000 ug/kg.

5. The pharmaceutical combination of claim 1, wherein said vaccine is in an amount of from about 1×10⁻⁸ g to about 1×10⁻⁴ g, and the immunomodulator compound of Formula A is in an amount of about 0.1-10 ug/kg.

6. The pharmaceutical combination of claim 1, wherein the immunomodulator compound of Formula A is in an amount of about 0.3-30 μg/kg.

7. The pharmaceutical combination of claim 1, comprising a composition including said vaccine and the immunomodulator compound of Formula A.

8. The pharmaceutical combination of claim 7, wherein said composition includes a pharmaceutically acceptable carrier.

9. A vaccination method comprising administering to an immunodeficient animal a pharmaceutical combination for enhancing vaccine effectiveness, said pharmaceutical combination comprising:
A) an immune response-triggering vaccine capable of stimulating production in an animal of antibodies to a disease-causing agent foreign to said animal; and
B) a vaccine effectiveness-enhancing amount of an immunomodulator compound of Formula A, which enhances production of said antibodies in said animal, in response to said vaccine;
C) wherein said vaccine and an immunomodulator compound of Formula A may be adminstered separately or together, wherein effectiveness of said vaccine in said animal is enhanced by an immunomodulator compound of Formula A; and
D) wherein the immunomodulator compound of Formula A is 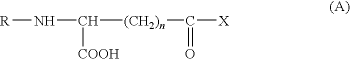
wherein n is 1 or 2, R is hydrogen, acyl, alkyl or a peptide fragment, and X is an aromatic or heterocyclic amino acid or a derivative thereof.

10. The method of claim 9, wherein said immunomodulator compound in SCV-07.

11. The method of claim 9, wherein said animal is human, and said vaccine is selected from the group consisting of Influenza vaccine, Hemophilus influenzae vaccine, Hepatitis A virus vaccine, Hepatitis B virus vaccine, Hepatitis C virus vaccine, Tuberculosis vaccine, Herpes-Zoster virus vaccine, Cytomegalovirus vaccine, Pneumococcal pneumonia vaccine, Meningococcal meningitis vaccine, Diphtheria vaccine, Tetanus vaccine, Rabies vaccine, Helicobacter pylori vaccine, polio vaccine and smallpox vaccine.

12. The method of claim 9, wherein said vaccine is in an amount of from about 1×10⁻⁹ g to about 1×10⁻³ g, and said an immunomodulator compound of Formula A is in an amount of about 0.001-1000 μg/kg.

13. The method of claim 9, wherein said vaccine is in an amount of from about 1×10⁻⁸ g to about 1×10⁻⁴ g, and said NGF is in an amount of from about 0.1-10 ug/kg.

14. The method of claim 9, wherein an immunomodulator compound of Formula A is in an amount of about 0.3-30 μg/kg.

15. The method of claim 9, wherein said vaccine is administered as a booster dose of vaccine.

16. The method of claim 15, wherein an immunomodulator compound of Formula A is administered about 3-4 days prior to said booster dose of vaccine.

17. The method of claim 15, wherein an immunomodulator compound of Formula A also is administered substantially concurrently with administration of said vaccine.

18. The method of claim 9, wherein said vaccine and an immunomodulator compound of Formula A are administered by injection.

19. The method of claim 9, wherein said immunomodulator compound of Formula A is administered orally.

20. A vaccination method comprising:
administering to a subject a first dose of an immune response-triggering vaccine capable of stimulating production in an subject of antibodies to a disease-causing agent foreign to said animal; and
administering to said subject either 1) a vaccine effectiveness-enhancing amount of an immunomodulator compound of Formula A which enhances production of said antibodies in said animal in response to said vaccine or 2) booster dose of said vaccine, along with a vaccine effectiveness-enhancing amount of an immunomodulator compound of Formula A, so as to enhance effectiveness of said vaccine in said subject, where an immunomodulator compound of Formula A, wherein the immunomodulator compound of Formula A is 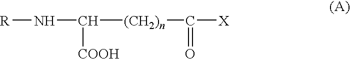
wherein n is 1 or 2, R is hydrogen, acyl, alkyl or a peptide fragment, and X is an aromatic or heterocyclic amino acid or a derivative thereof.

21. The method of claim 19, wherein said immunomodulator compound is SCV-07.

22. The method of claim 19, wherein said immunomodulator compound is administered with the vaccine.

23. The method of claim 19, wherein said immunomodulator compound is administered within a time period of immediately after the first dose and about two months after the first dose.
